(19) United States
(12) Patent Application Publication
(10) Pub. No.: US 2004/0241809 A1
(43) Pub. Date: Dec. 2, 2004

(54) Method for producing vitamin b12

(76) Inventors: Andreas Kuenkel, Neustadt (DE); Heiko Barg, Braunschweig (DE); Dieter Jahn, Wolfenbuttel (DE); Jan-Henning Martens, Braunschweig (DE); Martin Warren, Berkshire (GB)

Correspondence Address: Keil & Weinkauf, 1350 Connecticut Avenue N W, Washington, DC, 20036, US

(21) Appl. No.: 10/487,088

(22) PCT Filed: Aug. 20, 2002
(86) PCT No.: PCT/EP02/09271

(30) Foreign Application Priority Data
Aug. 22, 2001 (DE) 10141131.6
Oct. 11, 2001 (DE) 10150323.7

(51) Int. Cl. C12P 19/42; C12N 1/20

(52) U.S. Cl. 435/86; 435/252.31

(57) ABSTRACT

The present invention relates to a process for preparing vitamin B12 using Bacillus megaterium.

The present invention relates to a process for preparing vitamin B12 using Bacillus megaterium.

As long ago as the third decade of this century, vitamin B₁₂ was discovered indirectly through its effect on the human body by George Minot and William Murphy (Stryer, L., 1988, in Biochemie, fourth edition pp. 528-531, Spektrum Akademischer Verlag GmbH, Heidelberg, Berlin, N.Y.). Vitamin B₁₂ was purified and isolated for the first time in 1948, so that only eight years later, in 1956, its complex three-dimensional crystal structure was elucidated by Dorothy Hodgkin (Hodgkin, D. C. et al., 1956, Structure of Vitamin B₁₂. Nature 176, 325-328 and Nature 178, 64-70). The naturally occurring final products of the bio-synthesis of vitamin B12 are 5′-deoxyadenosylcobalamin (coenzyme B₁₂) and methylcobalamin (MeCbl), while vitamin B₁₂ is defined as cyanocobalamin (CNCbl) which is the form which is principally prepared and dealt with by industry. In the present invention, unless specifically stated, vitamin B12 always refers to all three analogous molecules.

The species B. megaterium was described for the first time by De Bary more than 100 years ago (1884). Although generally classified as a soil bacterium, B. megaterium can also be detected in various other habitats such as seawater, sediments, rice, dried meat, milk or honey. It is often associated with pseudomonads and actinomyces. B. megaterium is, like its close relation Bacillus subtilis, a Gram-positive bacterium and is distinguished inter alia by its relatively distinct size, which gives it its name, of 2×5 μm, a G+C content of about 38% and a very pronounced sporulation ability. Even minuscule amounts of manganese in the growth medium are sufficient for this species to carry out complete sporulation, an ability which is comparable only with the sporulation efficiency of some thermophilic bacilli. Because of its size and its very efficient sporulation and germination, diverse investigations have been carried out in the molecular bases of these processes in B. megaterium, so that more than 150 B. megaterium genes involved in its sporulation and germination have now been described.

Physiological investigations on B. megaterium (Priest, F. G. et al., 1988, A Numerical Classification of the Genus Bacillus, J. Gen. Microbiol. 134, 1847-1882) classified this species as an obligately aerobic, spore-forming bacterium which is urease-positive and Voges-Proskauer negative and is unable to reduce nitrate. One of the most prominent properties of B. megaterium is its ability to utilize a large number of carbon sources. Thus it utilizes a very large number of sugars and has been found, for example, in corn syrup, waste from the meat industry and even in petrochemical waste. In relation to this ability to metabolize an extremely wide range of carbon sources, B. megaterium can be equated without restriction with the pseudomonads (Vary, P. S., 1994, Microbiology, 40, 1001-1013, Prime time for Bacillus megaterium).

The advantages of the wide use of B. megaterium in the industrial production of a wide variety of enzymes, vitamins etc. are manifold. These include, firstly and certainly, the circumstance that plasmids transformed into B. megaterium prove to be very stable. This must be viewed in direct connection with the possibility which has now been established of transforming this species for example by polyethylene glycol treatment. Until a few years ago, this was still a major impediment to the use of B. megaterium as producer strain. The advantage of relatively well developed genetics must also be regarded in parallel with this, being exceeded within the Bacillus genus only by B. subtilis. Secondly, B. megaterium has no alkaline proteases, so that scarcely any degradation has been observed on production of heterologous proteins. It is additionally known that B. megaterium efficiently secretes products of commercial interest, as is utilized for example in the production of α- and β-amylase. In addition, the size of B. megaterium makes it possible to accumulate a large biomass before excessive population density leads to death. A further favorable circumstance of very great importance in industrial production using B. megaterium is the fact that this species is able to prepare products of high value and very high quality from waste and low-quality materials. This possibility of metabolizing an enormously wide range of substrates is also reflected in the use of B. megaterium as soil detoxifier able to break down even cyanides, herbicides and persistent pesticides. Finally, the fact that B. megaterium is completely apathogenic and produces no toxins is of very great importance, especially in the production of foodstuffs and cosmetics. Because of these many advantages, B. megaterium is already employed in a large number of industrial applications such as the production of α- and β-amylase, penicillin amidase, the processing of toxic waste or aerobic vitamin B₁₂ production (summarized in Vary, P. S., 1994, Microbiology, 40, 1001-1013, Prime time for Bacillus megaterium).

The use of Bacillus megaterium is of great economic interest because it has a number of advantages for use in the biotechnological production of various products of industrial interest. Optimization of the fermentation conditions, and molecular genetic modifications of B. megaterium are therefore of great commercial interest for the preparation of vitamin B12.

It is an object of the present invention to optimize the preparation of vitamin B12 using Bacillus megaterium.

We have found that this object is achieved by a process for preparing vitamin B12 using a culture containing Bacillus megaterium, in which the fermentation is carried out under aerobic conditions in a medium comprising at least cobalt and/or at least cobalt and 5-aminolevulinic acid.

It is possible in principle to employ for the purposes of the present invention all usual B. megaterium strains suitable as vitamin B12 producer strains. Vitamin B12 producer strains mean for the purposes of the present invention Bacillus megaterium strains or homologous microorganisms which have been altered by classical and/or molecular genetic methods so that their metabolic flux is increased in the direction of the biosynthesis of vitamin B12 or its derivatives (metabolic engineering). For example, one or more gene(s) and/or the corresponding enzymes in these producer strains which are located at key positions in the metabolic pathway which are crucial and subject to correspondingly complex regulation (bottleneck) have their regulation modified or are even deregulated. The present invention encompasses in this connection all previously known vitamin B12 producer strains, preferably of the genus Bacillus or homologous organisms. The strains which are advantageous according to the invention include, in particular, the strains DSMZ 32 and DSMZ 509 of B. megaterium.

In one variant of the process of the invention for preparing vitamin B12, cobalt is added in concentrations in the range from about 200 to 750 μM, preferably from about 250 to 500 μM.

In a further variant of the process of the invention, 5-aminolevulinic acid is added in concentrations in the range from about 200 to 400 μM, preferably of about 300 μM.

It is also possible according to the invention to improve the preparation of vitamin B12 using Bacillus megaterium in an advantageous manner by adding, for example, betaine, methionine, gutamate, dimethylbenzimidazole or choline, singly or in combinations.

The fermentation takes place according to the invention in medium containing glucose as C source. In a particularly advantageous variant of the process of the invention, the fermentation takes place in a medium containing glycerol as C source.

A higher cell density is generally reached on fermentation of Bacillus megaterium with glycerol as carbon source than with glucose. It is of interest in this connection that addition of cobalt together with 5-aminolevulinic acid under aerobic fermentation conditions leads to higher vitamin B12 production than in corresponding medium without additions.

This improved vitamin B12 production can be further increased according to the invention by converting the fermented Bacillus megaterium cells from aerobic to anaerobic growth conditions. The use of a culture medium containing glycerol, cobalt and 5-aminolevulinic acid has also proved particularly advantageous according to the invention in this case. The fermentation preferably takes place under aerobic conditions with the addition of about 250 μM cobalt; under anaerobic conditions it is advantageous to add about 500 μM cobalt.

Transferring the cultures from aerobic to anaerobic growth conditions makes it possible to combine high vitamin B12 contents with high cell densities.

The present invention thus also relates to a process in which the fermentation is carried out in a first step under aerobic conditions and in a second step under anaerobic conditions.

In a special variant of the present invention, the transition from aerobic to anaerobic fermentation takes place in the exponential growth phase of the aerobically fermentated cells. A further variant of the present invention provides a process in which the transition from aerobic to anaerobic fermentation takes place in the middle or at the end, preferably at the end, of the exponential growth phase of the aerobically fermented cells. In this connection, preference is given according to the invention to a process in which the transition from aerobic to anaerobic fermentation takes place as soon as the aerobic culture has reached its maximum optical density, but at least an optical density of about 2 to 3.

Anaerobic conditions mean for the purposes of the present invention those conditions which occur when the bacteria are transferred after aerobic culture into anaerobic bottles and fermented there. This means that the bacteria consume the oxygen present in the anaerobic bottles, and no further oxygen is supplied. These conditions may also be referred to as semi-anaerobic. Corresponding procedures are conventional laboratory practice and are known to the skilled worker. Comparable conditions also prevail when the bacteria are initially cultivated aerobically in a fermenter and then the oxygen supply is gradually reduced, so that semi-anaerobic conditions are eventually set up. In a special variant of the present invention, it is also possible for example to create strictly anaerobic conditions by adding reducing agents to the culture medium.

The fermentation medium contains according to the invention glucose as carbon source. An advantageous variant of the process of the invention comprises fermentation of B. megaterium on glycerol-containing medium. Further advantageous variants relate to a fermentation medium containing glucose or glycerol as C source and at least cobalt and/or cobalt and 5-aminolevulinic acid as addition. The two-stage process increases vitamin B12 production by a factor of at least 2.6 compared with production under completely aerobic conditions. If the medium contains glucose, cobalt and 5-aminolevulinic acid, it is possible by the two-stage fermentation to increase vitamin B12 production by a factor of at least 2.2 compared with production under completely aerobic conditions.

Production of vitamin B12 can also be increased even further according to the invention by employing genetically manipulated Bacillus megaterium strains. Such genetically modified bacterial strains can be produced by classical mutagenesis or targeted molecular biology techniques and appropriate selection methods. Starting points of interest for targeted genetic manipulation are, inter alia, points where the biosynthetic pathways leading to vitamin B12 branch, through which the metabolic flux can be deliberately guided in the direction of maximum vitamin B₁₂ production.

Targeted modifications of genes involved in the regulation of the metabolic flux also includes investigations and alterations of the regulatory regions upstream and downstream of the structural genes, such as, for example, the optimization and/or the exchange of promoters, enhancers, terminators, ribosome binding sites etc. The invention also encompasses improving the stability of the DNA, mRNA or of the proteins encoded by them, for example by reducing or preventing degradation by nucleases or proteases.

Also included in this connection according to the invention are polypeptides whose activity has been weakened or strengthened compared with the respective initial protein, for example by amino acid exchanges. The same applies to the stability of the enzymes of the invention in the cells, whose susceptibility to degradation by proteases has been increased or reduced for example.

The present invention also relates to corresponding polypeptides whose amino acid sequence has been modified such that they are desensitized toward compounds having regulatory activity, for example the final products of metabolism which regulate their activity (feedback desensitized).

The present invention also relates to a process for preparing vitamin B12 in which a Bacillus megaterium strain in which the cobA gene shows enhanced expression and/or is present in increased copy number is fermented. It is possible thereby to achieve an increase by a factor of at least 2.

Increased gene expression (overexpression) can be achieved by increasing the copy number of the appropriate genes. A further possibility is to modify the promoter region and/or regulatory region and/or the ribosome binding site located upstream of the structural gene in an appropriate manner for an increased rate of expression. Expression cassettes incorporated upstream of the structural gene can act in the same way. It is additionally possible by inducible promoters to increase the expression during vitamin B12 production.

Expression is likewise improved by measures to prolong the lifespan of the mRNA. The genes or gene constructs may either be present in plasmids in varying copy number or be integrated and amplified in the chromosome.

A further possibility is also for the activity of the enzyme itself to be increased or be enhanced by preventing degradation of the enzyme protein. A further alternative possibility is to achieve overexpression of the relevant genes by altering the composition of the media and management of the culture.

The present invention includes a gene structure comprising a nucleotide sequence of the cobA gene from B. megaterium coding for an S-adenosylmethionine-uroporphyrionogen III methyltransferase (SUMT) expressed under aerobic conditions, or parts thereof, and nucleotide sequences which are operatively linked thereto and have a regulatory function.

An operative linkage means the sequential arrangement for example of promoter, coding sequence, terminator and, where appropriate, further regulatory elements in such a way that each of the regulatory elements is able to carry out its proper function in the expression of the coding sequence. These regulatory nucleotide sequences may be of natural origin or be obtained by chemical synthesis. A suitable promoter is in principle any promoter which is able to control gene expression in the appropriate host organism. A possibility for this according to the invention is also a chemically inducible promoter able to control the expression of the genes subject to it in the host cell to a particular time. The β-galactosidase or arabinose system may be mentioned here by way of example.

A gene structure is produced by fusing a suitable promoter with at least one nucleotide sequence of the invention by conventional techniques of recombination and cloning as described, for example, in Sambrook, J. et al., Molecular Cloning: A Laboratory Manual, Cold Spring Harbor Laboratury, Cold Spring Harbor, N.Y. (1989).

Adaptors or linkers can be attached to the fragments for the joining together of the DNA fragments.

The invention also encompasses a vector comprising the nucleotide sequence of the cobA gene or parts thereof or a gene structure of the aforementioned type, and additional nucleotide sequences for selection, for replication in the host cell and/or for integration into the host cell genome. Suitable systems for the transformation and overexpression of genes of interest in B. megaterium are, for example, the plasmids pWH1510 and pWH1520, and the plasmid-free overexpression strain B. megaterium WH320, which are described by Rygus, T. et al. (1991, Inducible High-Level Expression of heterologous Genes in Bacillus megaterium using the Regulatory Elements of the Xylose-Utilization Operon, Appl. Microbiol. Biotechnol., 35, 594-599). Also advantageous according to the invention is the B. megaterium strain DSMZ509. However, the systems mentioned are not limiting for the present invention.

The present invention further relates to a transformed Bacillus megaterium strain for use in a process of the aforementioned type, which is distinguished in that it has enhanced expression and/or increased copy number of the nucleotide sequence of the gene cobA coding for an S-adenosylmethionine-uroporphyrionogen III methyltransferase.

Included in this connection according to the invention is also a transformed Bacillus megaterium strain which has in replicating form a gene structure or a vector of the aforementioned type comprising the cobA gene coding for an S-adenosylmethionine-uroporphyrionogen III methyltransferase from B. megaterium which is expressed under aerobic conditions. Expression of the cobA gene containing in the gene construct or vector of the aforementioned type may moreover take place both under aerobic and anaerobic conditions.

All B. megaterium strains suitable for vitamin B12 production are included according to the invention. These may also be genetically modified bacterial strains which have been or are produced by classical mutagenesis or targeted molecular biology techniques and appropriate selection methods.

Starting points of interest for targeted genetic manipulation are, inter alia, points where the biosynthetic pathways leading to vitamin B12 branch, through which the metabolic flux can be deliberately guided in the direction of maximum vitamin B₁₂ production.

One variant of the present invention includes a transformed B. megaterium strain which is distinguished in that it shows an increased vitamin B12 production according to the invention on fermentation under aerobic conditions compared with an untransformed strain, i.e. a strain which is not equipped with the cobA gene, a gene construct or vector of the aforementioned type.

In one variant of the process of the invention there is preferably fermentation of the transformed Bacillus megaterium strain in a medium containing glucose. A medium which contains glycerol as C source is particularly preferred. A further advantageous variant of the process of the invention includes fermentation in medium which, besides glucose or glycerol, additionally contains at least cobalt and/or cobalt and 5-amino-levulinic acid. Also advantageous according to the invention for preparing vitamin B12 is the two-stage fermentation of a transformed B. megaterium strain.

The present invention further relates to the use of the nucleotide sequence of the cobA gene coding for an S-adenosylmethionine-uroporphyrionogen III methyltransferase from B. megaterium for producing a transformed Bacillus megaterium strain of the aforementioned type. Also included according to the invention is the use of a transformed Bacillus megaterium strain of the aforementioned type for preparing vitamin B12.

The exemplary embodiments below serve to illustrate the present invention and have no limiting effect on the invention:

### 1. Bacterial Strains and Plasmids

All the bacterial strains and plasmids used in this study are listed in table 1 and table 2.

### 2. Buffers and Solutions

### 2.2 Minimal Media

| | | |
|---|---|---|
| Mopso minimal medium | | |
| Mopso (pH 7.0) | 50.0 | mM |
| Tricine (pH 7.0) | 5.0 | mM |
| MgCl₂ | 520.0 | μM |
| K₂SO₄ | 276.0 | μM |
| FeSO₄ | 50.0 | μM |
| CaCl₂ | 1.0 | mM |
| MnCl₂ | 100.0 | μM |
| NaCl | 50.0 | mM |
| KCl | 10.0 | mM |
| K₂HPO₄ | 1.3 | mM |
| (NH₄)₆Mo₇O₂₄ | 30.0 | pM |
| H₃BO₃ | 4.0 | nM |
| CoCl₂ | 300.0 | pM |
| CuSO₄ | 100.0 | pM |
| ZnSO₄ | 100.0 | pM |
| D-glucose | 20.2 | mM |
| NH₄Cl | 37.4 | mM |
| Titration reagent was KOH solution. | | |
| Salmonella typhimurium minimal medium | | |
| NaCl | 8.6 | mM |
| Na₂HPO₄ | 33.7 | mM |
| KH₂PO₄ | 22.0 | mM |
| NH₄Cl | 18.7 | mM |
| D-glucose | 20.2 | mM |
| MgSO₄ | 2.0 | mM |
| CaCl₂ | 0.1 | mM |
| 15 g/l agar-agar were added for solid media. | | |

### 2.2 Solutions for Protoplast Transformation of Bacillus megaterium

| | | |
|---|---|---|
| SMMP buffer | | |
| Antibiotic medium No. 3 (Difco) | 17.5 | g/l |
| Sucrose | 500.0 | mM |
| Na maleate (pH 6.5) | 20.0 | mM |
| MgCl₂ | 20.0 | mM |
| Titration reagent was NaOH solution. | | |
| PEG-P solution | | |
| PEG 6000 | 40.0% | (w/v) |
| Sucrose | 500.0 | mM |
| Na maleate (pH 6.5) | 20.0 | mM |
| MgCl₂ | 20.0 | mM |
| Titration reagent was NaOH solution. | | |
| cR5 top agar | | |
| Sucrose | 300.0 | mM |
| Mops (pH 7.3) | 31.1 | mM |
| NaOH | 15.0 | mM |
| L-proline | 52.1 | mM |
| D-glucose | 50.5 | mM |
| K₂SO₄ | 1.3 | mM |
| MgCl₂ × 6 H₂O | 45.3 | mM |
| KH₂PO₄ | 313.0 | μM |
| CaCl₂ | 13.8 | mM |
| Agar-agar | 4.0% | (w/v) |
| Casamino acids | 0.2% | (w/v) |
| Yeast extract | 10.0% | (w/v) |
| Titration reagent was NaOH solution. | | |

### 2.3. Solutions for Preparing Chromosomal Bacillus megaterium DNA

| | | |
|---|---|---|
| Saline EDTA (S-EDTA) | | |
| EDTA | 80.0 | mM |
| NaCl | 150.0 | mM |
| 0.1 × SSC solution | | |
| Trisodium citrate dihydrate | 1.5 | mM |
| NaCl | 50.0 | mM |
| adjust to pH 7.0 with HCl. | | |

### 2.3. Solutions and Markers for Agarose Gel Electrophoresis

| TAE buffer | | |
|---|---|---|
| Tris acetate (pH = 8.0) | 40.0 | mM |
| EDTA | 1.0 | mM |
| Sample buffer | | |
| Bromophenol blue | 350 | μM |
| Xylene cyanol FF | 450 | μM |
| Orange G | 0.25% | (w/v) |
| Sucrose in water | 115.0 | mM |
| Ethidium bromide solution | | |
| Ethidium bromide in water | 0.1% | (w/v) |

### GeneRuler DNA Ladder Mix

The marker contains the following fragments (in base pairs, bp): 10000, 8000, 6000, 5000, 4000, 3500, 3000, 2500, 2000, 1500, 1200, 1031, 900, 800, 700, 600, 500, 400, 300, 200, 100

### Lambda DNA/Eco91I (BstEII) Marker

Completely Eco91I-digested λ-DNA in water. The marker contains the following fragments (in base pairs, bp): 8453, 7242, 6369, 5687, 4822, 4324, 3675, 2323, 1929, 1371, 1264, 702, 224, 117

### 2.4 Solutions and Markers for SDS Polyacrylamide Gel Electrophoresis (SDS-PAGE)

| | | |
|---|---|---|
| Acrylamide stock solution | | |
| Acrylamide | 39.0% | (w/v) |
| N,N'-Methylenebisacrylamide | 1.0% | (w/v) |
| The solvent was water. | | |
| Stacking gel buffer | | |
| SDS | 0.4% | (w/v) |
| Tris-HCl (pH 6.8) | 1.5% | (w/v) |
| The solvent was water. | | |
| Resolving gel buffer | | |
| SDS | 0.4% | (w/v) |
| Tris-HCl (pH 8.8) | 1.5% | (w/v) |
| The solvent was water. | | |
| APS solution | | |
| Ammonium peroxodisulfate (APS) | 10.0% | (w/v) |
| The solvent was water. | | |
| Stacking gel [6% (w/v) for 5 minigels] | | |
| Acrylamide stock solution | 1.5 | ml |
| Stacking gel buffer | 2.5 | ml |
| Deion. water | 6.0 | ml |
| TEMED | 10.0 | μl |
| APS solution | 100.0 | μl |
| Resolving gel [12% (w/v) for 5 minigels] | | |
| Acrylamide stock solution | 6.0 | ml |
| Resolving gel buffer | 5.0 | ml |
| Deion. water | 9.0 | ml |
| TEMED | 20.0 | μl |
| APS solution | 200.0 | μl |
| Electrophoresis buffer | | |
| Glycine | 385.0 | mM |
| SDS | 0.1% | (w/v) |
| Tris-HCl (pH 8.8) | 50.0 | mM |
| The solvent was water. | | |
| Sample buffer | | |
| Glycerol | 40.0% | (w/v) |
| β-Mercaptoethanol | 2.0 | mM |
| SDS | 110.0 | mM |
| Bromophenol blue | 3.0 | mM |
| Tris-HCl (pH 6.8) | 100.0 | mM |
| Staining solution | | |
| Acetic acid | 10.0% | (v/v) |
| Coomassie Brilliant Blue (G-250 | 1.0 | g/l |
| The solvent was water. | | |
| Destaining solution | | |
| Ethanol | 30.0% | (v/v) |
| Glacial acetic acid | 10.0 | (v/v) |
| The solvent was water. | | |

| | |
|---|---|
| Dalton Mark VII | |
| (the relative molar mass Mᵣ is indicated in | |
| each case) | |
| α-Lactalbumin | 14200 |
| Trypsin inhibitor | 20100 |
| Trypsinogen | 24000 |
| Carbonic anhydrase | 29000 |
| Glyceraldehyde-3-phosphate | 36000 |
| dehydrogenase | |
| Ovalbumin | 45000 |
| Bovine serum albumin | 66000 |

### 2.5. Solutions for Protein Expression Experiments

| Disruption buffer | | |
|---|---|---|
| EDTA (pH 6.5) | 20.0 | mM |
| Na₃PO₄ | 100.0 | mM |
| Lysozyme | 5 | mg/ml |
| Titration reagent was H₃PO₄ solution. | | |

### 2.6. Solutions for Southern Blot Analysis

| Denaturing solution | | |
|---|---|---|
| NaOH | 500.0 | mM |
| NaCl | 1.5 | M |
| Neutralizing solution | | |
| Tris-HCl (pH 7.2) | 400.0 | mM |
| NaCl | 1.5 | M |
| 20 × SSC solution | | |
| Trisodium citrate dihydrate | 300.0 | mM |
| NaCl | 3.0 | M |
| adjust to pH 7.0 with HCl. | | |
| 10% blocking reagent | | |
| Milk powder in buffer 1 | 100 | g/l |
| Buffer 1 (maleic acid buffer) | | |
| Maleic acid (pH 7.5) | 100.0 | mM |
| NaCl | 150.0 | mM |
| NaOH | 200.0 | mM |
| adjust to pH 7.0 with HCl. | | |
| Buffer 2 | | |
| 10% strength blocking solution in buffer 1 | 100 | g/l |
| Buffer 3 (detection buffer) | | |
| Tris-HCl (pH 9.5) | 77.0 | mM |
| NaCl | 100.0 | mM |
| Washing buffer | | |
| Tween20 in buffer 1 | 3 | ml/l |
| Prehybridization solution | | |
| 20 × SSC | 250 | ml/l |
| N-lauroylsarcosine | 3.7 | mM |
| 10% strength SDS | 2 | ml/l |
| 20% strength blocking solution | 100 | ml/l |
| Hybridization solution | | |
| 20 × SSC | 250 | ml/l |
| N-lauroylsarcosine | 3.7 | mM |
| 10% strength SDS | 2 | ml/l |
| 20% strength blocking solution | 100 | ml/l |
| Probe solution | 5 | ml/l |

### 3. Media and Additions to Media

### 3.1. Media

Unless stated specially, the Luria-Bertani broth (LB) complete medium as described in Sambrook, J. et al. (1989, in Molecular cloning; a laboratory manual. 2^{nd} Ed., Cold Spring Harbor Laboratory Press, Cold Spring Harbor, N.Y.) was used. For solid media, 15 g of agar were additionally added per liter.

### 3.2. Additions

Additions such as carbon sources, amino acids, antibiotics or salts were either added to the media and autoclaved together or made up as concentrated stock solutions in water and sterilized, where appropriate by filtration. The substances were added to the media which had been autoclaved and cooled to below 50° C. With substances sensitive to light, such as tetracycline, care was taken to incubate in the dark. The final concentrations normally used were as follows:

| | | |
|---|---|---|
| ALA | 298 | μM |
| Ampicillin | 296 | μM |
| Casamino acids | 0.025% | (w/v) |
| CoCl₂ (in aerobic cultures) | 250 | μM |
| CoCl₂ (in anaerobic cultures) | 500 | μM |
| Cysteine | 285 | μM |
| Glucose | 22 | mM |
| Glycerol | 217 | mM |
| Lysozyme | 1 | mg/ml |
| Methionine | 335 | μM |
| Tetracycline (in solid media) | 23 | μM |
| Tetracycline (in liquid media) | 68 | μM |
| Xylose | 33 | mM |

### 4. Microbiological Techniques

### 4.1. Sterilization

Unless indicated otherwise, all the media and buffers were steam-sterilized at 120° C. and a gage pressure of 1 bar for 20 min. Thermally sensitive substances were sterilized by filtration (pore diameter of the filter 0.2 μm), and glassware was heat-sterilized at 180° C. for at least 3 h.

### 4.2. General Growth Conditions for Liquid Cultures of Bacteria

A sterile inoculating loop was used to take bacteria from an LB agar plate or from a glycerol culture and put them in the nutrient medium which contained an antibiotic if required.

Aerobic bacterial cultures were incubated in baffle flasks at 37° C. and at a rotational speed of 180 rpm. The incubation times were varied according to the desired optical densities of the bacterial cultures.

### 4.3. Conditions for Bacillus megaterium Growth

For the best possible aeration of aerobic cultures they were incubated in baffle flasks at 250 rpm and at 37° C. Anaerobic cultures were cultivated in a volume of 150 ml in 150 ml anaerobic bottles at 37° C. and 100 rpm. In both cases, care was taken to inoculate in the ratio 1:100 from overnight cultures, and to use constant conditions for the overnight cultures. In order to obtain higher yields of biomass under anaerobic conditions, B. megaterium cultures were preincubated aerobically and changed over at a desired density to anaerobic growth conditions. For this purpose, B. megaterium was initially incubated in baffle flasks at 37° C. and 250 rpm. In the middle of exponential growth or at the start of the stationary phase, the entire culture was transferred into a 150 ml anaerobic bottle and cultivation was continued at 37° C. and 100 rpm.

### 4.4. Bacterial Plate Cultures

A sterile inoculating loop was used to take bacteria from a glycerol culture and streak fractions on an LB agar plate, which was mixed with an appropriate antibiotic if required, so that individual colonies were visible on the plate after incubation at 37° C. overnight. If bacteria from a liquid culture were used, they were streaked on the LB agar plate using a Drygalski spatula and then incubated at 37° C. overnight.

### 4.5. Determination of Cell Density

The cell density of a bacterial culture was determined by measuring the optical density (OD) at 578 nm, with the assumption that an OD₅₇₈ of one is equivalent to a cell count of 1×10⁹ cells.

### 4.6. Storage of Bacteria

So-called glycerol cultures were prepared for prolonged storage of bacteria. For this purpose, 850 μl of a bacterial overnight culture were thoroughly mixed with 150 μl of sterile 85% glycerol and then stored at −80° C.

### 5. Molecular Biology Methods

The isolation of DNA and all techniques for restriction, Klenow and alkaline phosphatase treatment, sequencing, PCR etc. are routine laboratory practice and described in the standard work for molecular biology methods by Sambrook, J. et al. (1989, in Molecular cloning; a laboratory manual. 2nd Ed., Cold Spring Harbor Laboratory Press, Cold Spring Harbor, N.Y.).

### 5.1. Cloning of cobA in PWH1520

The cloning and expression vector used was pWH1520 (Rygus et al., 1991). The pBR322 derivative has a tetracycline resistance and an ampicillin resistance, and the elements important for replication in E. coli and Bacillus ssp. This system is thus amenable to all cloning techniques established in E. coli and can be simultaneously used for gene expression in B. megaterium. The vector contains the B. megaterium xylA and xylR genes of xyl operon with the relevant regulatory sequences (Rygus et al., 1991). The xylA gene codes for xylose isomerase, while xylR codes for a regulatory protein which exerts strong transcriptional control on the xylA promoter. The xylA gene is repressed by XylR in the absence of xylose. On addition of xylose there is an approximately 200-fold induction through derepression of xylA. A polylinker of the plasmid in the xylA reading frame makes it possible to fuse target genes with xylA, which are then likewise under the strong transcriptional control of XylR. It is moreover possible to choose between the alternatives of forming a transcription or translation fusion, because the xylA reading frame is still completely intact upstream from the polylinker.

To construct a cobA overexpression clone, the known sequence (Robin et al., 1991) was taken from the B. megaterium genome. PCR primers permitting translation fusion of CobA with xylose isomerase were derived therefrom. The ribosome binding sequence of the xylA gene within the expression vector pWH1520 is thus utilized. An SpeI cleavage site and a BamHI cleavage site were integrated into the PCR primer, and the desired cobA sequence was amplified from genomic B. megaterium DNA by PCR. Both the amplified gene sequence, and the overexpression vector pWH1520 were then each cut with SpeI and BamHI, and the cohesive ends produced in this way were ligated. It was possible to isolate clones which, after digestion with SpeI and BamHI, showed inserts of the desired size. The integrity of the cloned DNA was checked by complete DNA sequence determination. The cloning strategy is depicted diagrammatically in FIG. 5.

### 5.2. Production of Competent Cells

Competent E. coli and B. megaterium cells were produced by cultivating 500 ml liquid cultures with LB medium until the OD₅₇₈ was 0.5-1. The culture was cooled on ice and centrifuged (4000×g; 15 min; 4° C.). The cell sediment was thoroughly resuspended in sterile deionized water, centrifuged (4000×g; 8 min; 4° C.), again washed with sterile deionized water and recentrifuged (4000×g; 8 min; 4° C.). The sediment was washed with 10% strength (v/v) glycerol solution and then centrifuged (4000×g; 8 min; 4° C.), and the sediment was resuspended in the minimum amount of 10% strength (v/v) glycerol solution. The competent E. coli and B. megaterium cells were immediately used for the transformation.

### 5.3. Transformation of Bacteria by Electroporation

The transformation took place by electroporation using a gene pulser with connected pulse controller (BioRad). For this purpose, 140 μl each of competent E. coli or B. megaterium cells and 1 μg of plasmid DNA were transferred into a transformation cuvette and exposed in the gene pulser to a field strength of 12 kV/cm at 25 μF and a parallel resistance of 200 Ω.

For the subsequent regeneration, the transformed cells were, immediately after the transformation, incubated in 1 ml of LB medium in a thermoshaker at 37° C. for half an hour, in the case of B. megaterium for one hour. Various volumes of the mixtures were then streaked on LB plates with appropriate addition of antibiotics and incubated at 37° C. overnight.

### 5.4. Protoplast Transformation of Bacillus megaterium

### Protoplast Preparation

50 ml of LB medium were inoculated with 1 ml of an overnight culture of B. megaterium and incubated at 37° C. At an OD₅₇₈ of 1 the cells were centrifuged (10 000×g; 15 min; 4° C.) and resuspended in 5 ml of freshly prepared SMMP buffer. Addition of lysozyme in SMMP buffer was followed by incubation of the suspension at 37° C. for 60 min, monitoring protoplast formation under the microscope. The cells were harvested by centrifugation (3000×g; 8 min; Rt) and then the cell sediment was carefully resuspended in 5 ml of SMMP buffer, and the centrifugation and washing steps were carried out a second time. It was then possible, after addition of 10% (w/v) glycerol, to divide the protoplast suspension into portions and freeze them at −80° C.

### Transformation

500 μl of the protoplast suspension were mixed with 0.5 to 1 μg of DNA in SMMP buffer, and 1.5 ml of PEG-P solution were added. After incubation at Rt for 2 min, 5 ml of SMMP buffer were added and carefully mixed, and the suspension was centrifuged (3000×g; 5 min; Rt). Immediately thereafter, the supernatant was removed and the scarcely visible sediment was resuspended in 500 μl of SMMP buffer. The suspension was incubated at 37° C., shaking gently, for 90 min. Then 50-200 μl of the transformed cells were mixed with 2.5 ml of cR5 top agar and put onto LB-agar plates which contained antibiotics suitable for selection. Transformed colonies were visible after incubation at 37° C. for one day.

### 5.5. Quantitative Vitamin B₁₂ Analysis

For quantitative determination of vitamin B₁₂, samples were taken from B. megaterium cultures in various growth phases. After determination of the OD₅₇₈, the cells were separated from the medium by centrifugation (4000×g; 15 min; 4° C.). Washing with 40 ml of isotonic NaCl solution was followed by recentrifugation (4000×g; 15 min; 4° C.). The resulting cell sediments, and the removed media, were subsequently freeze dried. S. typhimurium metE cysG double mutants were incubated on methionine- and cysteine-containing minimal medium at 37° C. overnight, scraped off the plate and washed with 40 ml of isotonic NaCl solution. After centrifugation, the cell sediment was resuspended in isotonic saline. The washed bacterial culture was carefully mixed with 400 ml of cysteine-containing minimal medium agar at 47-48° C.

10 μl of the B. megaterium samples which had been resuspended in sterile deionized water and boiled in a water bath for 15 min were put on the cooled plates and incubated at 37° C. for 18 h. The diameters of the Salmonella colonies which grow are then proportional to the vitamin B₁₂ content in the B. megaterium samples applied. The vitamin B₁₂ content in the investigated samples was deduced by comparison with a calibration plot constructed by adding 0.01, 0.1, 1, 10 and 40 μmol of vitamin B₁₂. This standard method allows small amounts of vitamin B₁₂ to be detected rapidly and very reproducibly in biological materials.

### 5.6. Preparation of Chromosomal Bacillus megaterium DNA

To obtain chromosomal DNA, 150 ml of LB medium were inoculated with B. megaterium and incubated at 37° C. and 250 rpm overnight. The culture was centrifuged (4000×g; 10 min; 4° C.) and the bacterial sediment was resuspended in 13 ml of S-EDTA. A spatula tip of lysozyme which had previously been dissolved in 1 ml of S-EDTA was added to the suspension. 800 μl of 25% strength SDS solution were also added to the solution and incubated at 37° C. in a thermoshaker for 30 min. After one hour at 65° C., the solution was mixed with 3.2 μl of 5M sodium perchlorate and 20 ml of chloroform/isoamyl alcohol mixture (24:1). The mixture was shaken at 0° C. for 30 min and then centrifuged (12 000×g; 10 min; 4° C.). The upper DNA-containing phase was carefully removed, transferred into a 50 ml graduated cylinder and slowly covered by a layer of 30 ml of ethanol. The chromosomal DNA precipitating at the phase boundary was wound onto a glass rod by a rotating motion and unwound into 5 ml of 0.1×SSC solution.

### 6. Protein Expression

### 6.1. Overexpression of S-adenosyl-L-methionine-uroporphyrinogen III methyltransferase (SUMT) in Bacillus megaterium

150 ml of LB medium were inoculated with 1.5 ml of a B. megaterium overnight culture and incubated aerobically at 37° C. Bacteria containing the expression plasmid pWH1520-cobA were selected by adding tetracycline. After an OD₅₇₈ of 0.3 was reached, the xyl promoter of the expression plasmid was induced by adding 0.5% (w/v) xylose. A sample of 2 OD₅₇₈ equivalents was taken before the induction and each hour after the induction. The removed samples were centrifuged (12 000×g; 3 min; Rt) and the sedimented cells were resuspended in 40 μl of disruption buffer. The suspension was then incubated at 37° C. for 30 min. 20 μl of the disrupted material were mixed with 5 μl of SDS-PAGE sample buffer and, after boiling in a water bath for 15 minutes, centrifuged at 15 000 rpm for 30 min (8000×g; 10 min; Rt). The supernatant was analyzed by an SDS-PAGE.

### KEY TO THE FIGURES

Bacterial strains and plasmids as shown in tables 1 and 2 were employed.

Table 1: Bacterial strains used

Table 2: Plasmids used

The present invention is explained further by means of the figures below.

FIG. 1 shows the vitamin B₁₂ production by B. megaterium DSM509 under aerobic growth conditions in Mopso minimal medium. The vitamin B₁₂ content in μg per liter of bacterial culture is indicated for glucose without additions (1), glucose with addition of 250 μM CoCl₂ (2), glucose with addition of 298 μM ALA and 250 μM CoCl₂ (3), glycerol without additions (4), glycerol with addition of 250 μM CoCl₂ (5), glycerol with addition of 298 μM ALA and 250 μM CoCl₂ (6).

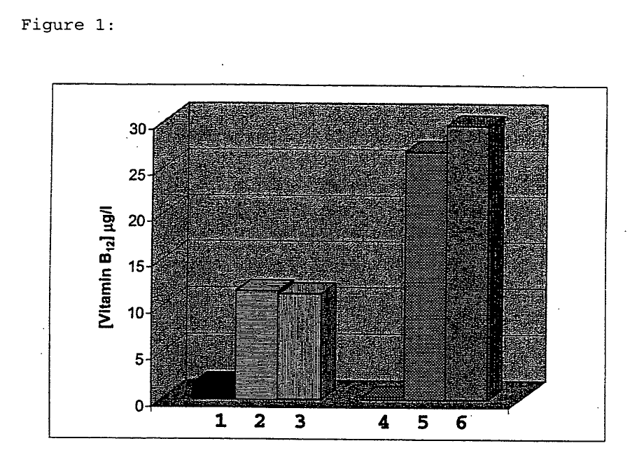

FIG. 2 shows a comparison of the vitamin B₁₂ production by B. megaterium DSM509 under aerobic growth conditions and with transfer to anaerobic growth conditions, in each case with addition of 298 μM ALA and 250 μM CoCl₂ (aerobic) or 500 μM CoCl₂ (anaerobic). The vitamin B₁₂ content in μg per liter of bacterial culture is indicated for cultures with glucose under aerobic conditions (1), glucose transferred in the middle of the exponential phase (OD₅₇₈=3.0) (2), glucose transferred at the end of the exponential phase (OD₅₇₈=5.9) (3), glycerol under aerobic conditions (4), glycerol transferred in the middle of the exponential phase (OD₅₇₈=4.7) (5), glycerol transferred at the end of the exponential phase (OD₅₇₈=12.0) (6).

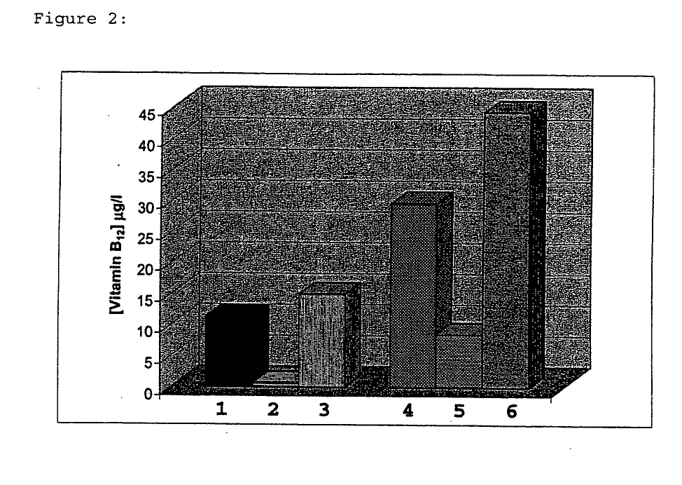

FIG. 3 shows the vitamin B₁₂ production by the transformed B. megaterium strain DSM509 pWH1520-cobA compared with B. megaterium DSM509 under aerobic growth conditions in LB medium. The vitamin B₁₂ content in μg per liter of bacterial culture is indicated for:

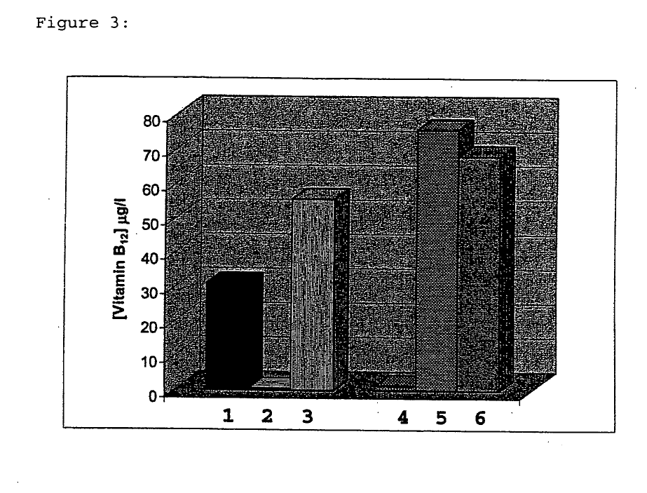

DSM509: without additions (1), with addition of 250 μM COCl₂ (2), with addition of 298 μM ALA and 250 μM CoCl₂ (3).

DSM509-pWH1520-cobA: without additions (4), with addition of 250 μM CoCl₂ (5), with addition of 298 μM ALA and 250 μM CoCl₂ (6).

FIG. 4 shows a comparison of the vitamin B₁₂ production by B. megaterium DSM509 pWH1520-cobA in LB medium under aerobic (1) and anaerobic (2) growth conditions and with transfer to anaerobic growth conditions (3). The transfer took place at the end of the exponential phase at an OD₅₇₈ of 6.9. The vitamin B₁₂ content in μg per liter of bacterial culture is indicated. All cultures contained addition of 298 μM ALA and 250 μM CoCl₂.

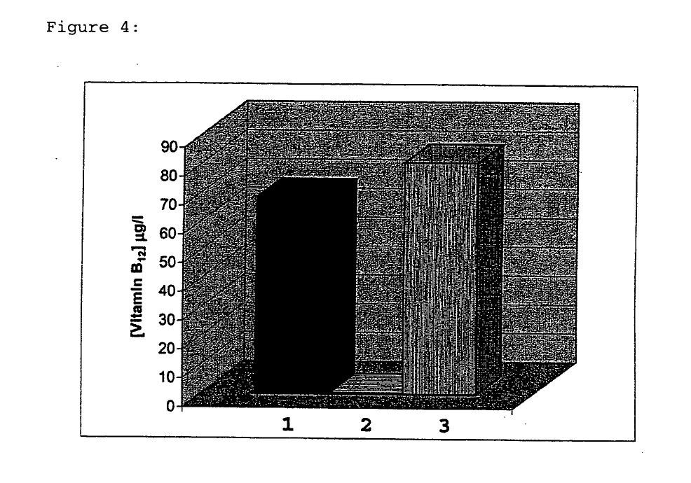

FIG. 5 shows a diagrammatic representation of the cloning of the cobA gene from B. megaterium into the overexpression vector pWH1520. The gene amplified by PCR and the vector were each cut with SpeI and BamHI, and the resulting cohesive ends were ligated to give a xylA-cobA translation fusion within the newly produced overexpression vector pWH1520-cobA.

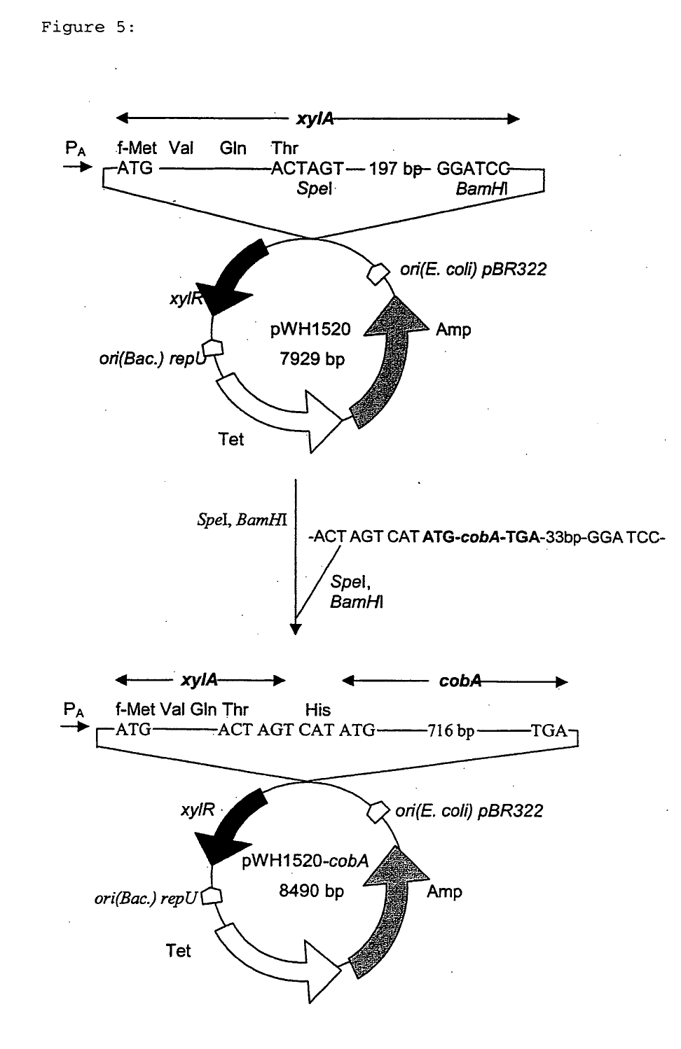

### Tables and Figures

TABLE 1

| Strain | Description | Reference/source |
|---|---|---|
| Escherichia | | |
| coli | | |
| DH5α | Fλ⁻supE44Δ(argF-lac) | Sambrook et al., |
| | U169φ89dlacZΔM15hsdR17recA | 1989 |
| | 1endA1gyrA96thi-1relA1 | |
| Bacillus | | |
| megaterium | | |
| DSMZ32 | Wild type | DSMZ* |
| DSMZ509 | Vitamin B₁₂ producer | DSMZ* |
| WH320 | DSMZ319 lac | Rygus et al., 1991 |
| Salmonella | | |
| typhimurium | | |
| AR 3612 | Leu⁺ cysG metE, Sm^{r} | Raux et al., 1996 |

*DSMZ: Deutsche Sammlung von Mikroorganismen und Zellkulturen, Brunswick

TABLE 2

| Plasmid | Description | Reference/source |
|---|---|---|
| pWH1520 | Cloning and expression | Rygus et al., 1991 |
| | vector for Bacillus spp., | |
| | Ap^{r}, Tc^{r} | |
| pWH1520-cobA | 716 bp BamHI-SpeI fragment | this study |
| | with B. megaterium cobA in | |
| | pWH1520 | |

1. A process for preparing vitamin B12 using a culture comprising Bacillus megaterium, wherein the fermentation is carried out under aerobic conditions in a medium comprising at least cobalt and/or at least cobalt and 5-aminolevulinic acid.

2. A process as claimed in claim 1, wherein cobalt is added in concentrations in the range from about 200 to 750 μm.

3. A process as claimed in claim 1, wherein 5-aminolevulinic acid is added in concentrations in the range from about 200 to 400 μm.

4. A process as claimed in claim 1, wherein the fermentation is carried out in a medium comprising glycerol as C source.

5. A process as claimed in claim 1, wherein the fermentation is carried out in a first step under aerobic and in a second step under anaerobic conditions.

6. A process as claimed in claim 1, wherein the transition from aerobic to anaerobic fermentation takes place in the exponential growth phase of the aerobically fermented cells.

7. A process as claimed in claim 1, wherein the transition from the aerobic to the anaerobic fermentation takes place in the middle or at the end, preferably at the end, of the exponential growth phase of the aerobically fermented cells.

8. A process as claimed in claim 1, wherein the transition from the aerobic to the anaerobic fermentation takes place as soon as the aerobic culture has reached its maximum optical density or at least an optical density in the range from about 3 to 12.

9. A process as claimed in claim 1, wherein a Bacillus megaterium strain in which the cobA gene shows enhanced expression and/or is present in increased copy number is fermented.

10. A transformed Bacillus megaterium strain for use in a process as claimed in claim 1, which shows enhanced expression and/or increased copy number of the nucleotide sequence of the cobA gene coding for an S-adenosylmethionine-uroporphyrionogen III methyltransferase from B. megaterium.

11-12. (canceled).

13. A process for preparing vitamin B12, said process comprising transforming a Bacillus megaterium strain with a cobA gene coding for an S-adenosylmethionine-uroporphyrionogen III methyltransferase from B. megaterium and fermenting said strain under aerobic conditions.
